(19) United States
(12) Patent Application Publication
(10) Pub. No.: US 2015/0320845 A1
(43) Pub. Date: Nov. 12, 2015

(54) System and Method for Modulation of Cardiac Tissue

(71) Applicant: The Regents of the University of California, Oakland, CA (US)

(72) Inventor: Kalyanam Shivkumar, Los Angeles, CA (US)

(21) Appl. No.: 14/706,312

(22) Filed: May 7, 2015

Related U.S. Application Data
(60) Provisional application No. 61/989,890, filed on May 7, 2014.

(51) Int. Cl. A61K 38/48; A61B 18/00

(52) U.S. Cl. CPC A61K 38/4886; A61B 18/00; C12Y 304/24007

(57) ABSTRACT

The present invention provides compositions, systems, and methods for modulation of cardiac tissue. In one embodiment, the invention provides for bioenzymatic ablation of cardiac tissue. In certain embodiments, the present invention provides for application of collagenase or other therapeutic agents to digest targeted cardiac tissue.

### CROSS-REFERENCE TO RELATED APPLICATIONS

This application claims priority to U.S. Provisional Patent Application No. 61/989,890 filed May 7, 2014, the contents of which are incorporated by reference herein in their entirety.

### STATEMENT REGARDING FEDERALLY SPONSORED RESEARCH OR DEVELOPMENT

This invention was made with government support under R01HL084261, awarded by the National Institutes of Health (NIH). The government has certain rights in the invention.

### BACKGROUND OF THE INVENTION

Catheter ablation is an established strategy for treating and preventing recurrent ventricular arrhythmias (Calkins et al., 2000, J Am Coll Cardiol, 35: 1905-1914; Kuck et al., 2010, Lancet, 375: 31-40; Stevenson et al., 2008, Circulation, 118: 2773-2782). A common approach to ventricular tachycardia (VT) ablation is targeting reentrant circuits through activation mapping and entrainment mapping, when the VT is hemodynamically tolerated (de Bakker et al., 1988, Circulation, 77: 589-606; Stevenson et al., 1993, Circulation, 88: 1647-1670). VT episodes that result in hemodynamic instability are generally targeted by substrate modification during sinus rhythm. Electroanatomic mapping (EAM) helps delineate normal myocardial regions from abnormal ones, and identifies low-amplitude, delayed multicomponent electric activity, referred to as isolated late potentials (LPs). LPs may indicate areas of slow conduction that represent targets for catheter ablation of VT (Marchlinski et al., 2000, Circulation, 101: 1288-1296; Soejima et al., Circulation, 2001, 104: 664-669; Cesario et al., 2007, Heart rhythm, 4: S44-S50).

Radiofrequency (RF) energy delivery is most commonly used for catheter ablation (Cesario et al., 2007, Heart rhythm, 4: S44-S50; Nakagawa et al., 1995, Circulation, 91: 2264-2273), however, alternative ablation energy sources such as cryoablation (Deisenhofer et al., 2010, Circulation, 122: 2239-2245; Skanes et al., 2000, Circulation, 102: 2856-2860), high-intensity focused ultrasound (HIFU) (Strickberger et al., 1999, Circulation, 100: 203-208), laser (Pfeiffer et al., 1996, Circulation, 94: 3221-3225; Ware et al., 1999, Circulation, 99: 1630-1636) and microwave (Thomas et al., 1999, J Cardiovasc Electrophysiol, 10: 72-78; Whayne et al., 1994, Circulation, 89: 2390-2395) have been studied. Chemical ablation by ethanol and coil embolization of a small coronary artery branch have also been reported (Brugada et al., 1989, Circulation, 79: 475-482; Tholakanahalli et al., 2013, Heart rhythm, 10: 292-296).

However, there remains a need in the art for targeted biological ablation of VT. The present invention satisfies this unmet need.

### SUMMARY OF THE INVENTION

In one aspect, the present invention provides a method of modulating a region of cardiac tissue. The method comprises contacting at least a portion of the region of cardiac tissue with an effective amount of at least one therapeutic agent selected from the group consisting of a protease, a collagenase, a lipase, and a detergent. In certain embodiments, the method reduces the size of border zones of scar tissue, thereby at least partially homogenizing the scar tissue. In one embodiment, the method treats arrhythmia.

In one embodiment, the at least one therapeutic agent removes fat from the region. In one embodiment, the region includes surviving myocytes. In one embodiment, the region is a border zone region. In one embodiment, the region is an area of low-amplitude, delayed multicomponent electric activity.

In one embodiment, the therapeutic agent comprises a collagenase solution. In one embodiment, the collagenase acts upon type IV collagen. In one embodiment, the collagenase is purified collagenase. In one embodiment, the concentration of collagenase in the collagenase solution is between 0.001-1%. In one embodiment, the amount of collagenase in the collagenase solution is between 1-1000 U/mL.

In one embodiment, the at least one therapeutic agent is delivered via a catheter. In one embodiment, the catheter is an ablation catheter.

In one embodiment, the method comprises using electroanatomical mapping to define the region prior to contacting at least a portion of the region with the therapeutic agent.

In one aspect, the present invention provides a system for modulating a region of cardiac tissue. The system comprises at least one therapeutic agent selected from the group consisting of a protease, a collagenase, a lipase, and a detergent, and a device for delivering the at least one therapeutic agent to a target site of the cardiac tissue. In one embodiment, the system ablates at least a portion of the region of cardiac tissue. In one embodiment, the system treats arrhythmia. In one embodiment, the at least one therapeutic agent removes fat from the region.

In one embodiment, the at least one therapeutic agent comprises a collagenase solution. In one embodiment, the device comprises one or more catheters, where the collagenase solution is delivered through a lumen of the one or more catheters.

In one embodiment, the collagenase solution is contained in one or more reservoirs positioned at the distal end of the device. In one embodiment, the one or more reservoirs comprise an absorbent material at least partially saturated with the collagen solution. In one embodiment, the absorbent material comprises a sponge or sponge like material. In one embodiment, the device comprises one or more catheters connected to the one or more reservoirs for delivery of the therapeutic agent to the one or more reservoirs.

In one aspect, the present invention provides an ablation kit. The kit comprises at least one therapeutic agent selected from the group consisting of a protease, a collagenase, a lipase, and a detergent, and a device for delivering the at least one therapeutic agent to a target site of cardiac tissue. In one embodiment, the at least one therapeutic agent comprises a collagenase solution.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1L, are a set of images depicting collagenase application in vitro. Shown are trichrome elastic von Giessen images of tissue soaked into (FIG. 1A) HEPES (control), (FIG. 1B) Purified collagenase (CLSPA) solution (400 U/mL), (FIG. 1C) collagenase type 2 (CLG-2) 0.4% and (FIG. 1D) collagenase type 4 (CLG-4) 0.4% (Scale bar, 1 mm). Scar tissue is stained blue. FIG. 1E, FIG. 1F, FIG. 1G, and FIG. 1H indicate corresponding higher-power fields of collagenous fibrotic scar area from FIG. 1A, FIG. 1B, FIG. 1C, and FIG. 1D respectively, and adjacent myocardium in FIG. 1I, FIG. 1J, FIG. 1K, and FIG. 1L respectively (magnification×10; scale bar, 100 μm). Arrows indicate regions digested, and scale bars on the tissue indicate depth of myocardial digestion.

FIG. 2, comprising FIG. 2A through FIG. 2D, are a set of images depicting collagenase delivery strategies. (FIG. 2A), Image of collgenase type 4 (CLG-4) application in subject 3 using small cellulose sponges (2×2 mm). (FIG. 2B), Bipolar voltage map before and after CLG-4 application in subject 3. Arrows indicate CLG-4 application site. (FIG. 2C), Cellulose sponge (0.5×1 cm) tied to the tip of an irrigation catheter. Arrows indicate CLG-4 solution connected to irrigation lumen. (FIG. 2D), Bipolar voltage map of right ventricular inferior epicardium before and after CLG-4 application in subject 5. Dense scar lesion with voltage <0.5 mV is shown in gray. Arrows indicate CLG-4 application site.

FIG. 3, comprising FIG. 3A through FIG. 3C, depicts the results of experiments demonstrating the change in low voltage area after collagenase application. (FIG. 3A), Bipolar voltage map before and after collagenase type 4 (CLG-4) application. Dense scar lesion with voltage <0.5 mV was delineated in gray areas. Arrows indicate CLG-4 application site. (FIG. 3B), Quantification of scar area in the animals with CLG-4 selectively applied (n=5). (FIG. 3C), Quantification of border zone and dense scar distribution in scar area.

FIG. 4, comprising FIG. 4A through FIG. 4C, depicts the results of experiments demonstrating that collagenase application eliminates late potentials. (FIG. 4A), Bipolar voltage map and isolated late potential (LP) distribution before (top) and after (bottom) collagenase type 4 (CLG-4) application. Arrows on the before application image and at the bottom right of the after application image indicate LPs in the scar area. The grey arrowheads indicate actual LPs on the electrogram. The arrows on the left and top right of the after application image indicate LP eliminated points. The white arrows denote a LP that remained after CLG-4 application. (FIG. 4B), Quantification of total collected points from electro-anatomical maps before and after CLG-4 application. (FIG. 4C), Quantification of the number of LPs before and after CLG-4 application.

FIG. 5, comprising FIG. 5A through FIG. 5D, are a set of images depicting the histopathological analyses after collagenase application. Representative images of trichrome elastic von Giessen staining of scar tissue after collagenase type 4 (CLG-4) application in (FIG. 5A) subject 1 and (FIG. 5C) subject 5 (scale bar, 250 μm). (FIG. 5B) and (FIG. 5D) indicate corresponding higher-power fields of (FIG. 5A) and (FIG. 5C), respectively (scale bar 100 μm). Arrowheads in FIG. 5A indicate connective tissue loosening. Black double-sided arrows indicate the approximate range of digestion lesion.

FIG. 6 is a table of the procedural details of the in vitro experiments. Values for digestion depth are listed as mean±SEM. CLSPA=Purified collagenase; CLG-2=Collagenase type 2; CLG-4=Collagenase type 4. *P<0.05 when compared to CLG-2.

FIG. 7 is a table of the procedural details of collagenase application strategies. RCA=right coronary artery; LAD=left anterior descending artery; RV=right ventricle; LV=left ventricle; Epi=epicardium. kg=kilogram; min=minute; mm=millimeter.

FIG. 8 is an illustration of an exemplary device of the invention.

FIG. 9 is an illustration of a reservoir of an exemplary device of the invention.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, or ±5%, or ±1%, or ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compositions, systems, and methods for biological manipulation of cardiac tissue. In certain embodiments, the invention is used as a therapy for the treatment of arrhythmia in a subject. For example, in one embodiment, the invention is used to eliminate abnormal cardiac tissue, including, for example regions characterized by slow conductance, low voltage, and/or the presence of late potentials (LPs). In one embodiment, the invention provides the elimination of border zones (BZs), which are regions of tissue where myocardial scars are adjacent to normal tissue. The BZ regions represent regions of slow conduction and, in certain instances, are sources of ventricular tachycardia.

The present invention is not limited to treating any particular type of arrhythmia. Rather, any type of arrhythmia known in the art to be caused by one or more regions of abnormal cardiac tissue may be treated by way of this invention. Exemplary forms of arrhythmia include, but are not limited to atrial tachycardia, atrial flutter, atrial fibrillation, premature atrial contractions, supraventricular tachycardia, AV nodal reentrant tachycardia, junctional rhythm, junctional tachycardia, premature ventricular contractions, ventricular tachycardia, accelerated idioventricular rhythm, ventricular fibrillation, AV block, and the like.

In one embodiment, the invention provides a composition comprising at least one therapeutic agent. In certain embodiments, the therapeutic agent is any biological or chemical compound that modulates the activity or function of cardiac cells or cardiac tissue. Exemplary therapeutic agents include, but are not limited to proteins, enzymes, peptides, nucleic acids, vectors, small molecules, hormones, cells, antibodies, antibody fragments, detergents and the like. In one embodiment, the therapeutic agent is any agent or compound that can degrade or digest components of cardiac tissue. For example, the therapeutic agent may comprise any agent or compound that degrades or digests cells, proteins, extracellular matrix proteins, fat, and the like. In certain embodiments, the therapeutic agent is an enzyme. In one embodiment, the enzyme is a protease. In another embodiment, the enzyme is a lipase. In another embodiment, the therapeutic agent is a detergent that dissolves fat in the tissue. In certain embodiments, the composition comprises more than one therapeutic agent.

The present invention is partly based upon the discovery that application of collagenase effectively reduces the area of BZs. Further, collagenase application has minimal impact on normal myocardial tissue. The composition of the invention can thus be applied to a region of damaged or abnormal cardiac tissue to reduce or remove such tissue. In certain embodiments, the composition induces the homogenization of a scar. However, the present invention is not limited to collagenase, but rather encompasses any therapeutic agent. In certain embodiments, the composition comprises a therapeutic agent that digests one or more regions of target tissue. Aside from collagenase, other compounds that would act to digest regions of target tissue include, but are not limited to, clostripain, trypsin, trypsin-like enzyme, aminopeptidase, caseinase, secondary proteases, lipases, detergents, and the like.

The present invention encompasses any type of collagenase, including, but not limited to type 1 collagenase, type 2 collagenase, type 3 collagenase, type 4 collagenase, and a combination thereof. In one embodiment, the collagenase acts to degrade type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, or a combination thereof.

In certain embodiments, the composition comprises purified collagenase. In another embodiment, the composition comprises collagenase in combination with one or more additional therapeutic agents that may be useful in treating or removing the abnormal cardiac tissue. Such additional therapeutic agents include, but are not limited to proteases, clostripain, trypsin, trypsin-like enzyme, aminopeptidase, caseinase, secondary proteases, lipases, detergents, and the like.

In certain embodiments, the composition is comprises a collagenase solution. In one embodiment, the concentration of collagenase is between about 0.001% to about 1%. In another embodiment, the concentration of collagenase is between about 0.01% to about 0.5%. In another embodiment, the concentration of collagenase is between about 0.02% to about 0.4%.

In one embodiment, the amount of collagenase in the collagenase solution is between about 1 U/mL to about 100 U/mL.

The collagenase solution comprises collagenase dissolved in any suitable solvent, including but not limited to water, saline, buffered solutions, and the like. In one embodiment, the solvent comprises HEPES.

In certain embodiments, the composition of the invention comprises a peptide. In one embodiment, the composition comprises an isolated nucleic acid encoding a peptide. In certain embodiments, the composition comprises a vector comprising an isolated nucleic acid encoding the peptide. In one embodiment, the composition comprises a cell comprising an isolated nucleic acid encoding the peptide.

As described elsewhere herein, exemplary peptides of the invention include collagenases, proteases, clostripain, trypsin, trypsin-like enzyme, aminopeptidase, caseinase, secondary proteases, lipases, and the like.

In one embodiment, the invention includes variants of the peptides of the invention. In one embodiment, variants differ from naturally-occurring peptides by conservative amino acid sequence differences or by modifications which do not affect sequence, or by both. For example, conservative amino acid changes may be made, which although they alter the primary sequence of the peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

In a further embodiment, the peptide of the invention comprise D-, L-, and unnatural isomers of amino acids.

As known in the art the “similarity” between two peptides is determined by comparing the amino acid sequence and its conserved amino acid substitutes of one polypeptide to a sequence of a second peptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

Variants of suitable peptides of the invention can also be expressed. Variants may be made by, for example, the deletion, addition, or alteration of amino acids that have either (i) minimal influence on certain properties, secondary structure, and hydropathic nature of the polypeptide or (ii) substantial effect on one or more properties of the peptide mimetics of the invention.

Variants may also include, for example, a peptide conjugated to a linker or other sequence for ease of synthesis, purification, identification, or therapeutic use (i.e., delivery) of the peptide.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

The peptides of the invention may be prepared by standard chemical or biological means of peptide synthesis. Biological methods include, without limitation, expression of a nucleic acid encoding a peptide in a host cell or in an in vitro translation system.

Included in the invention are nucleic acid sequences that encode the peptide of the invention. Accordingly, subclones of a nucleic acid sequence encoding a peptide of the invention can be produced using conventional molecular genetic manipulation for subcloning gene fragments, such as described by Sambrook et al., Molecular Cloning: A Laboratory Manual, Cold Springs Laboratory, Cold Springs Harbor, N.Y. (2012), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for a particular activity.

Biological preparation of a peptide of the invention involves expression of a nucleic acid encoding a desired peptide. An expression cassette comprising such a coding sequence may be used to produce a desired peptide for use in the method of the invention.

In the context of an expression vector, the vector can be readily introduced into a host cell, e.g., mammalian, bacterial, yeast or insect cell by any method in the art. Coding sequences for a desired peptide of the invention may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

The expression vector can be transferred into a host cell by physical, biological or chemical means, as described in detail elsewhere herein.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Examples of biological methods to prepare the peptides of the present invention may utilize methods provided in US Patent Application Publication No. US2009/0069241, which is incorporated herein in its entirety.

To ensure that the peptide obtained from either chemical or biological synthetic techniques is the desired peptide, analysis of the peptide composition can be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

In certain embodiments, the therapeutic agent of the composition comprises an isolated nucleic acid. For example, in one embodiment, the isolated nucleic acid encodes a peptide of the invention. In certain embodiments, the isolated nucleic acid allows for local expression of the peptide at a treatment site. For example, the isolated nucleic acid may be used to genetically modify one or more cells at or near the treatment site. The genetically modified cell can then express the peptide of the invention, thereby providing local delivery of a therapeutic agent.

The nucleic acid sequences coding for the desired peptide can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a nucleic acid of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity.

In brief summary, the expression of natural or synthetic nucleic acids encoding a therapeutic peptide is typically achieved by operably linking a nucleic acid encoding the peptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a peptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate nucleic acid and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the composition of the invention comprises a substrate comprising a therapeutic agent of the invention (e.g. collagenase). The present invention is not limited to any particular substrate, but rather encompasses any suitable substrate known in the art. For example, in certain embodiments, the substrate is a patch, sponge, bandage, gauze, or the like. In one embodiment, the substrate is a tissue engineered substrate. For example, in one embodiment, the substrate is a biocompatible substrate comprising one or more biopolymers, synthetic polymers, or a combination thereof. Such substrates may include, for example, hydrogels, electrospun scaffolds, or other tissue engineering substrates known in the art.

In one embodiment, the substrate is adsorbed with solution comprising one or more therapeutic agents. In another embodiment, the substrate is embedded with one or more therapeutic agents. For example, in certain embodiments, one or more therapeutic agents are covalently or non-covalently bound to the interior or surface of the substrate.

In certain embodiments, the composition of the invention is designed for transient or temporary application to the cardiac tissue. In another embodiment, the composition is designed for mid-term or permanent application to the cardiac tissue. For example, in one embodiment, the substrate of the composition degrades or is adsorbed into the tissue.

The invention also encompasses the use of pharmaceutical compositions of the invention or salts thereof to practice the methods of the invention. Such a pharmaceutical composition may consist of at least one compound or conjugate of the invention or a salt thereof in a form suitable for administration to a subject, or the pharmaceutical composition may comprise at least one compound or conjugate of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be used to prepare an aqueous or oily suspension or solution by addition of an aqueous or oily vehicle thereto. Each of these formulations may further comprise one or more of dispersing or wetting agent, a suspending agent, and a preservative.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The present invention provides a system for bioenzymatic or chemical modulation of cardiac tissue. For example, in certain embodiments, the system provides for a bioenzymatic or chemical ablation of targeted cardiac tissue.

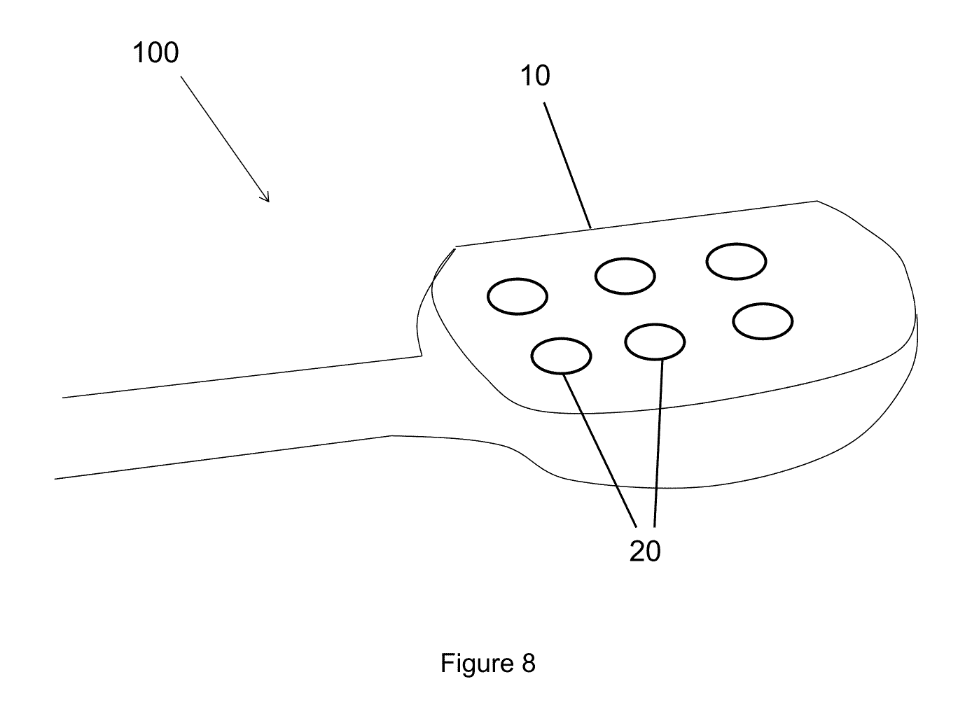

As depicted in FIG. 8, in certain embodiments, the system comprises a device 100, deliverable to the targeted tissue, and at least one therapeutic agent. In one embodiment, the therapeutic agent is deliverable to the target site via one or more catheters of device 100. For example, in one embodiment, the therapeutic agent is delivered to the target site through the lumen of the one or more catheters.

In another embodiment, the therapeutic agent is contained within, or deliverable to, one or more reservoirs 20, positioned for example at the distal end 10 of device 100. Distal end 10 of device 100 may be placed in contact with a target region of cardiac tissue to deliver the therapeutic agent to the target region. In one embodiment, reservoir 20 is a hollow chamber. In one embodiment, reservoir 20 comprises a sponge or other material adsorbed, impregnated, or embedded with the therapeutic agent. For example, in one embodiment, device 100 comprises one or more catheters and a substrate affixed to or positioned at the distal end 10 of device 100, where the substrate comprises the therapeutic agent. In certain embodiments, the therapeutic agent of the system is collagenase or other compound that functions to degrade or digest the target tissue.

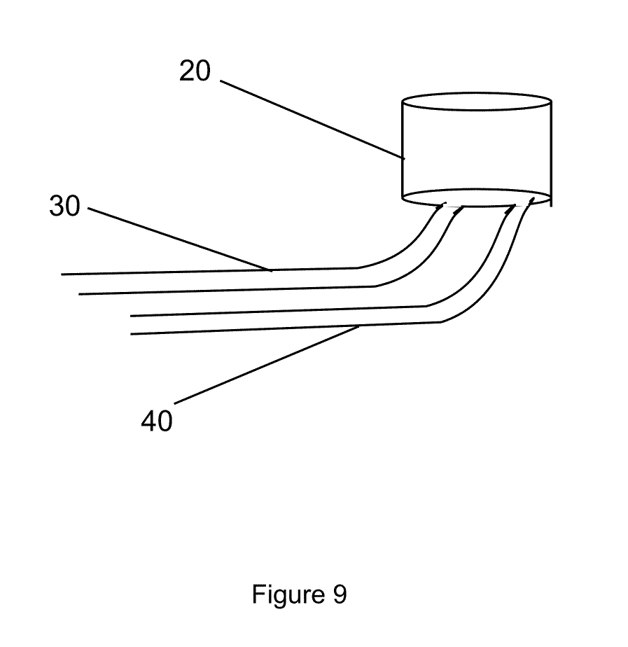

As depicted in FIG. 9, in certain embodiments, device 100 comprises one or more input lines 30 and one or more output lines 40, connected to the one or more reservoirs 30 positioned at the distal end 10 of device 100. Each input line 30 and output line 40 comprises a hollow tube having a lumen through which the therapeutic agent may travel. For example, the therapeutic agent may be delivered through input line 30 to reservoir 20, and drained through output line 40, if needed. In certain embodiments, each reservoir 20 is accessed by a single tube or lumen, which can serve as both input line 30 and output line 40. In certain embodiments, input line 30 and/or output line 40 may comprise one or more valves, timers, or sensors that control delivery or drainage of the therapeutic agent to and from reservoir 30.

In one embodiment, the system comprises a pump, used to deliver the therapeutic agent through device 100. For example, the pump may be connected to one or more input lines 30 and/or output lines 40, to deliver the therapeutic agent to one or more reservoirs 20 positioned at the distal end 10 of device 100 of the system.

In one embodiment, the system comprises a control unit which may control pump parameters, the timing of the delivery of a therapeutic agent, duration of treatment, or the like. The control unit may be programmed directly or remotely by a health care provider. In certain embodiments, the control unit comprises electrical or mechanical sensors which can be used to determine treatment parameters.

In one embodiment, the present invention provides a method for modulating the function of cardiac tissue. For example, in certain embodiments, the invention provides a method for treating arrhythmia in a subject in need thereof. As discussed elsewhere herein, the invention encompasses treatment of any type of arrhythmia. In one embodiment, the method comprises contacting a region of cardiac tissue with at least one therapeutic agent.

In one embodiment, the method comprises the bioenzymatic ablation of damaged or abnormal cardiac tissue. For example, in one embodiment, the method of the invention includes the application of a therapeutic agent to a region of damaged or abnormal cardiac tissue in order to remove or reduce the size of such tissue. The target tissue may be any type of cardiac tissue including epicardium, myocardium, and endocardium.

The method of the invention comprises administration of any suitable therapeutic agent. Exemplary therapeutic agents include, but are not limited to proteins, enzymes, peptides, nucleic acids, vectors, small molecules, hormones, cells, antibodies, antibody fragments, detergents and the like. In one embodiment, the therapeutic agent is any agent or compound that can degrade or digest components of cardiac tissue. For example, the therapeutic agent may comprise any agent or compound that degrades or digests cells, proteins, extracellular matrix proteins, fat, and the like. In certain embodiments, the therapeutic agent is an enzyme. In one embodiment, the enzyme is a protease. In another embodiment, the enzyme is a lipase. In another embodiment, the therapeutic agent is a detergent that dissolves fat in the tissue. In certain embodiments, the method comprises contacting the region of cardiac tissue with more than one therapeutic agent.

In certain embodiments, the method comprises administering a therapeutic agent that digests one or more regions of target tissue. Aside from collagenase, other compounds that would act to digest regions of target tissue include, but are not limited to, clostripain, trypsin, trypsin-like enzyme, aminopeptidase, caseinase, secondary proteases, lipase, detergents, and the like.

In one embodiment, the method comprises identifying the particular region or regions of cardiac tissue to be targeted for ablation. The identification of target tissue may be done by any suitable method known in the art. For example, in certain embodiments, identification of the target tissue is done by suitable imaging techniques which provide a visual indication of target tissue. Exemplary imaging techniques include, but are not limited to cardiac CT, cardiac PET, cardiac MRI, myocardial perfusion imaging, and the like. In one embodiment, identification of the target tissue is done by mapping the functional activity of the cardiac tissue. For example, in certain embodiments, the method comprises electroanatomic mapping. In one embodiment, the method comprises determining a voltage map of the cardiac tissue which can identify areas of low-voltage, which are characteristic of scar and BZ. In certain embodiments, the method comprises generating of electrograms depicting the electrical activity of one or more regions of the cardiac tissue. Exemplary systems which may be used for electroanatomic mapping of the tissue of the subject include the NavX patch system and Ensite array catheter system (Ensite, St. Jude Medical).

In one embodiment, the present method comprises administering an effective amount of one or more therapeutic agents (e.g. collagenase) to the target tissue. In certain embodiments, the method induces the targeted digestion of normal and scarred tissue in a targeted BZ, which thus homogenizes the scar tissue. In one embodiment, the method comprises applying a substrate comprising one or more therapeutic agents directly on the target tissue. In one embodiment, the substrate is applied to the target tissue during a surgical procedure. In another embodiment, the substrate is delivered to the target tissue via a catheter. As described elsewhere herein, the substrate may be any suitable biocompatible substrate known in the art. In one embodiment, the method comprises transiently applying the substrate to the target issue thereby applying the embedded one or more therapeutic agents to the target tissue. In another embodiment, the method comprises permanently affixing the substrate to the target tissue. In certain embodiments, the substrate is configured to degrade or adsorb into the tissue of the subject. In another embodiment, the method comprises delivering a catheter to the target solution and applying a solution comprising the one or more therapeutic agents to the target tissue via the distal end of the catheter. The catheter may be delivered to the target tissue via any suitable route known in the art. For example, in one embodiment, a catheter or needle is inserted through the pericardium to access the cardiac tissue. In another embodiment, a catheter is steered to the target tissue via one of the blood vessels that access the heart. For example, a catheter can be delivered via the aorta, vena cava, or other suitable vessels that access the heart. In one embodiment, the method comprises inter-ventricular transseptal access, where a catheter is delivered from one ventricle to the other ventricle through an access site created though the inter-ventricular septum.

In certain embodiments, the method of the invention encompasses administration of any type of collagenase, including, but not limited to type 1 collagenase, type 2 collagenase, type 3 collagenase, type 4, collagenase, and a combination thereof. In one embodiment, the administered collagenase acts to degrade type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, or a combination thereof.

In certain embodiments, the method comprises administration of purified collagenase. In another embodiment, the method comprises administration of collagenase in combination with one or more additional therapeutic agents that may be useful in treating or removing the abnormal cardiac tissue. Such additional therapeutic agents include, but are not limited to proteases, clostripain, trypsin, trypsin-like enzyme, aminopeptidase, caseinase, secondary proteases, lipases, detergents, and the like.

In certain embodiments, the method comprises administering collagenase solution. In one embodiment, the concentration of collagenase is between about 0.001% to about 1%. In another embodiment, the concentration of collagenase is between about 0.01% to about 0.5%. In another embodiment, the concentration of collagenase is between about 0.02% to about 0.4%.

In one embodiment, the amount of collagenase in the collagenase solution is between about 1 U/mL to about 100 U/mL.

The collagenase may be applied for any suitable amount of time. For example, as described elsewhere herein, in certain embodiments, the method comprises transient or temporary application of collagenase. In one embodiment, the time that collagenase is applied is between about 5 seconds to about 2 hours. In another embodiment, the time that collagenase is applies is between 1 minute to about 1 hour.

In certain embodiments, the method comprises inhibiting the activity of the applied collagenase. For example, in one embodiment, a collagenase antagonist or inhibitor is applied to the subject or to the target tissue of the subject. In certain embodiments, the inhibition of collagenase is done after it is determined that collagenase activity is no longer needed. For example, in certain embodiments, the use of various imaging modalities described herein or functional mapping described herein is used to determine that the target tissue has been ablated.

In certain embodiments, the method comprises a single administration of collagenase to the target tissue. In another embodiment, the method comprises repeated administration of collagenase to the target tissue. The particular dose and treatment regimen may be determined on an individualized basis depending on the disease severity, age, gender, and health of the subject.

The therapeutic composition of the invention may be administered to the subject either prior to or after a diagnosis of arrhythmia. Further, several divided dosages, as well as staggered dosages may be administered daily or sequentially, or the dose may be continuously infused, or may be a bolus injection. Further, the dosages of the therapeutic compositions may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The composition may be administered to a subject as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, every 5 days week, every week, every two weeks, and the like. It will be readily apparent to one skilled in the art that the frequency of administration of the various combination compositions of the invention will vary from subject to subject depending on many factors including, but not limited to, age, type and severity of the disease or disorder to be treated, gender, overall health, type of subject, and other factors. Thus, the invention should not be construed to be limited to any particular dosage regime and the precise dosage and composition to be administered to any subject will be determined by the attending physical taking all other factors about the subject into account.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the composition in dosage unit form for ease of administration and uniformity of dosage. Dosage unit form as used herein refers to physically discrete units suited as unitary dosages for the subjects to be treated; each unit containing a predetermined quantity of therapeutic composition calculated to produce the desired therapeutic effect in association with the required pharmaceutical vehicle. The dosage unit forms of the invention are dictated by and directly dependent on (a) the unique characteristics of the therapeutic composition and the particular therapeutic effect to be achieved, and (b) the limitations inherent in the art of compounding/formulating such a therapeutic composition for the treatment of a disease in a subject.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Electrical Homogenization of Ventricular Scar by Application of Collagenase

Collagen subtypes I and III are abundant in myocardial scars (Luther et al., 2012, Circ Res, 110: 851: 856; Cleutjens et al., 1995, Am J Pathol, 147: 325-338; Lopez et al., 2010, Circulation, 121: 1645-1654; Uusimaa et al., 1997, Circulation, 96: 2565-2572) and contribute to regions of slow conduction and electrical instability, known substrates for VT initiation (Rutherford et al., 2012, Circ Res, 111: 301-311; Ursel et al., 1985, Circ Res, 56: 436-451. Since border-zones (BZ) and LPs are characterized by surviving myocytes interspersed with scar (Nakahara et al., 2010, Heart Rhythm, 7: 1817-1824), it was examined whether collagenase (CLG) application induce focal digestion of both normal and scar tissue in BZs, thus homogenizing the scar and abolishing LPs in the process. The experiments presented herein evaluate and characterize the feasibility of topical CLG application in homogenizing myocardial scars, and to assess the impact of CLG on LPs assessed by electro-anatomical mapping. A porcine infarct model, which closely resembles human myocardial scars, was utilized. Optimal collagenase subtype and concentration were determined by in vitro experiments, and applied topically in vivo to epicardial scar regions.

The materials and methods employed in these experiments are now described.

Myocardial Infarct Induction

Myocardial infarcts (MI) were created in six female Yorkshire pigs (30 to 35 kg). Following a 12-hour fasting period, the animals were sedated with intramuscular injection of 1.4 mg/kg Telazol, and were intubated. Ventilation was achieved with an endotracheal tube connected to a ventilator (Summit Medical, Bend, Oreg.). General anesthesia was maintained with inhaled 1.5% to 2.5% isoflurane. Analgesia was maintained with buprenorphine (0.3 mg) intravenously hourly. Femoral arterial and venous access were obtained, and lidocaine (2.0 mg/kg), esmolol (1.0 mg/kg) and unfractionated heparin (10,000 units) were given intravenously. Under fluoroscopic guidance, myocardial infarctions were created in the left circumflex (LCX) (n=1), right coronary (RCA) (n=2), and left anterior descending coronary arteries (LAD) (n=3). An Amplatz-type guide catheter was placed in the left main coronary artery or the RCA. A 0.018 mm guide wire (HT BMW Universal, Abbott vascular, IL) was inserted into the left coronary artery or the RCA. A 2.5-3.5 mm angioplasty balloon catheter (Fox sv, Abbott vascular, IL) was advanced over a guidewire and inflated in the mid-LCX/LAD or in the mid RCA. Thirty seconds after balloon inflation, a 10 mL suspension of sterile saline containing 3 to 5 mL polystyrene microspheres (Polybead® 90.0 μm, Polysciences, PA) was injected through the central lumen of the balloon catheter. Electrocardiogram and arterial pressure were monitored continually during infarction and recovery. Acute infarction was confirmed by ST segment elevation in the electrocardiogram leads. Five minutes after microsphere injection, the balloon catheter was removed. Animals were extubated and observed with continuous electrocardiogram monitoring until able to ambulate without assistance. Animals were observed without additional anti-arrhythmic drugs administered until terminal CLG-application experiments.

In Vitro Testing of Collagenase Subtypes

Three types of clostridial CLGs [Type 2 collagenase (CLG-2); Type 4 collagenase (CLG-4); and purified collagenase (CLSPA); Worthington Biochemical Corporation, NJ] were evaluated to identify the optimal CLG subtype, and concentration for scar homogenization (Hoppe et al., 1998, Circulation, 97: 55-65; Masson-Pevet et al., 1976, J Mol Cell Cardiol, 8: 747-757; Todor et al., 2002, Am J Physiol Heart Circ Physiol, 283: H990-995; Zhou et al., 2000, Am J Physiol Heart Circ Physiol, 279: H429-436). CLG-2, and CLG-4 were each tested at 6 concentrations (0.025%, 0.05%, 0.1%, 0.15%, 0.2% and 0.4%) while CLSPA was tested at 6 dilutions (25 U/mL, 50 U/mL, 100 U/mL, 150 U/mL, 200 U/mL and 400 U/mL). The pieces of myocardium were obtained from a LCX infarct pig. The infarct was grossly evident on inspection, and was cut out into 20 equal pieces. The pieces of myocardium were subsequently histologically confirmed to contain scar, BZ and normal myocardium. A chemical buffer solution (HEPES, Sigma-Aldrich, Mo.) was used to dissolve each CLG subtype. The CLG solutions were adjusted to pH 7.4-7.5 for experimentation. Pieces of tissue soaked into HEPES medium served as controls. The solutions containing tissue were incubated at 37° C. for 24 hours. Histopathological analyses were performed to evaluate tissue digestion.

Collagenase Application In Vivo

Open chest surgery and topical application of CLG was performed in four animals. CLG-4 in 0.8% was used for each experiment. Cellulose sponge (small patch, 2×2 mm; large patch 3×5 cm; Cellulose sponge, Ningbo Kingyn, China) adsorbed with CLG-4 solution was placed onto the epicardial surface of the scar. The sponges were carefully placed within the low voltage areas where LPs were identified during EAM. The size of CLG-4 application area was decided based on the EAM results. During CLG application, the body was kept warm using a heating blanket. After confirming the feasibility of topical CLG application using the open chest approach, one additional animal was studied using a closed chest topical approach (Sosa et al., 1996, J Cardiovasc Electrophysiol, 7: 531-536). This involved devising a commercially available electrophysiologic ablation catheter, with a piece of cellulose sponge sutured tightly onto it (4 mm SafireBLU, St. Jude Medical, MN). The facilitated visualization of the catheter on the EAM system, for precise delivery of CLG-4 to selected areas. After CLG-4 application in vivo, all animals (total n=5) subjected to CLG application were euthanized. Histopathological analyses were performed to evaluate scar homogenization.

Electroanatomic Mapping

Electroanatomic bipolar voltage mapping of epicardium was performed during sinus rhythm on the animals subjected to CLG application. The animals were heparinized during mapping and CLG application (3000 units unfractionated heparin intravenously every hour). The NavX patch system (EnSite, St. Jude Medical, MN) was used in the animal with closed-chest topical approach, while an EnSite array catheter system (EnSite, St. Jude Medical, MN) was used in the animals with open-chest approach. EnSite array was chosen to avoid the impact of air on impedance as assessed by NavX patch system. Data from both NavX patch and EnSite array systems were analyzed using NavX Velocity software (St. Jude Medical, MN). A duodecapolar catheter (Livewire, 2-2-2 mm spacing, St. Jude Medical, MN) and/or a 4 mm tip mapping catheter (SafireBLU, St. Jude Medical, MN) were used for epicardial mapping. During epicardial mapping, at least 500 points were collected for each pre- and post-CLG map.

All points on the interior projection >8 mm from the geometry (exterior projection for epicardial mapping) were considered to represent insufficient contact and excluded from the voltage map. Bipolar electrograms were band pass filtered between 30 and 300 Hz and displayed at a sweep speed of 100 mm/s. Three-dimensional bipolar electroanatomic maps were displayed with dense scar (DS) defined as <0.5 mV, scar BZ from 0.51 to 1.50 mV, and total low-voltage area as <1.5 mV. Total low-voltage and DS areas were measured offline. Electrogram timing was measured manually during retrospective offline analysis. Late potential was defined as any low-voltage electrogram (<1.5 mV) with a distinct onset after the QRS showing double or multiple components separated by a >20 msec isoelectric interval (Nakahara et al., J Am Coll Cardiol, 55: 2355-2365). Epicardial mapping was performed before and after CLG application. Total scar area, BZ area, DS area and the number of LPs (the number of mapped sites showing LPs) were quantified to compare between pre and post CLG application.

Histopathological Analysis

After euthanization, hearts were immediately explanted and rinsed thoroughly with cold saline 4° C. Subsequently cold 10% buffered formalin was flushed down the coronary arteries repeatedly. These samples were then placed in 10% buffered formalin for 24 to 48 hours, and then into 70% ethanol after rinsing in dH₂O. The sections were embedded in paraffin and cut in 5 micron-thick sections, and then stained with hematoxylin and eosin and trichrome-elastic van Gieson stains. Slides were digitally scanned for measurement of lesion depth (Aperio XT, Aperio Technologies, Vista, Calif.).

Statistical Analysis

The exact permutational version of the nonparametric Wilcoxon signed-rank test was used to compute P values for paired comparisons of hemodynamic data, total scar, DS, and BZ areas before and after CLG application.

The exact nonparametric Mann-Whitney U test was used for unpaired comparisons of digestion depth. Data are displayed as dot plots. A P value <0.05 was considered significant. Analyses were performed with the use of SPSS (version 19.0) statistical software (SPSS, Chicago, Ill.).

Catheter stability and reproducibility of local electrograms (including LPs) was verified by sampling repeatedly at each location at different time intervals. Two observers analyzed the morphology and timing of these potentials. In cases in which a measurement or electrogram was subject to interpretation, a consensus between the two observers was reached.

The results of the experiments are now described

### Determination of Optimal Collagenase Subtype

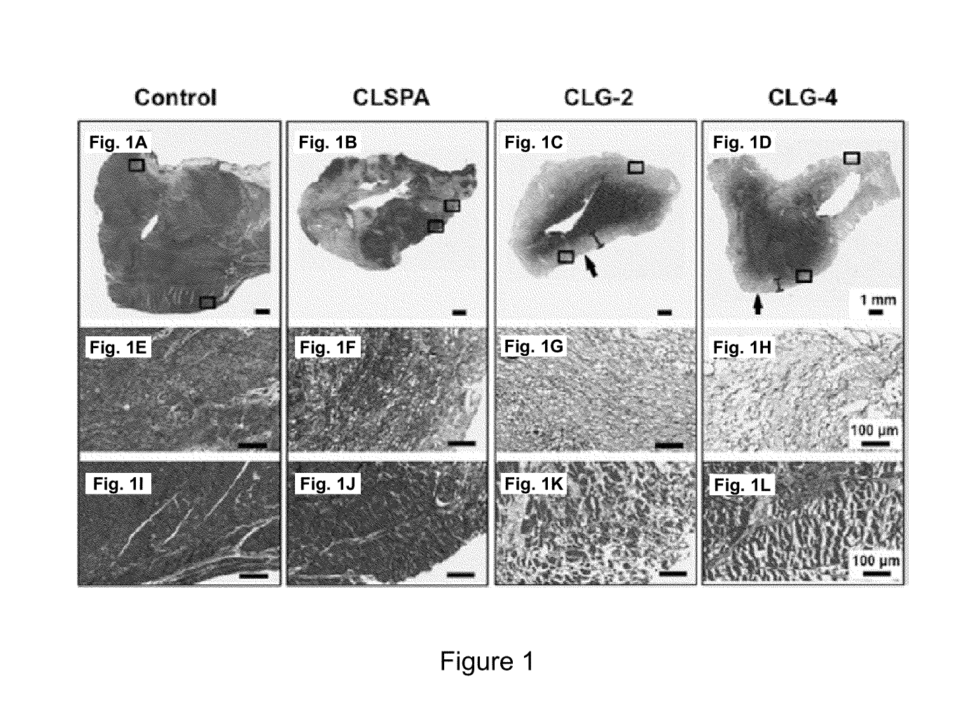

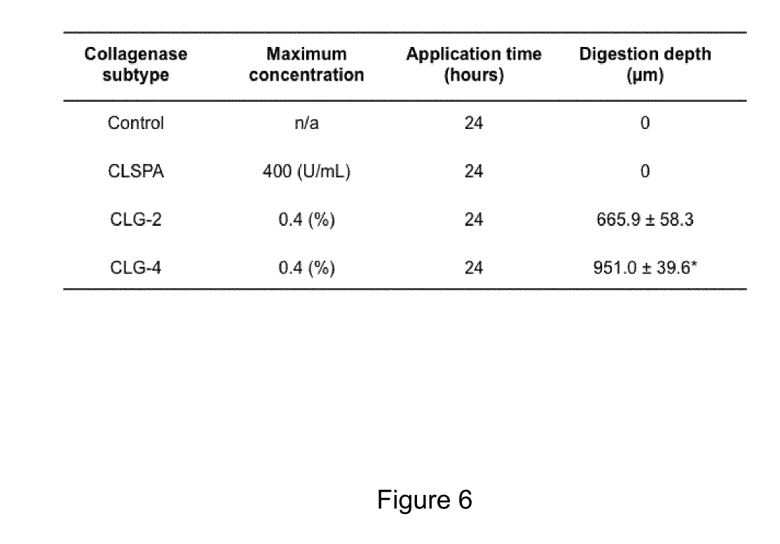

In vitro scar digestion was performed from adjacent blocks of myocardial tissue containing scar, BZ, and normal appearing myocardium. The procedural details are shown in FIG. 6. Three CLG subtypes were tested as previously described, CLG-2, CLG-4, and CLSPA. Concentrations tested were 0.025%, 0.05%, 0.1%, 0.15%, 0.2% and 0.4% for CLG-2 and CLG-4, and 25 U/mL, 50 U/mL, 100 U/mL, 150 U/mL, 200 U/mL and 400 U/mL for CLPSA. FIG. 1 shows histologic results from digestion experiments. FIG. 1A shows control tissue block soaked in HEPES solution, while FIGS. 1E and 1I demonstrate high power views of the scar and normal appearing myocardium respectively from FIG. 1A. As shown in FIG. 1B, FIG. 1F, and FIG. 1J, CLSPA (even at the highest concentration of 400 U/mL), showed minimal digestion of the scar. However, the effects of CLG-2 (FIG. 1C, FIG. 1G, and FIG. 1K) and CLG-4 (FIG. 1D, FIG. 1H, and FIG. 1L) can be appreciated easily at doses of 0.4% for both CLG-2 and CLG-4. Below this dose, minimal digestion of scar or myocardium occurred. The effect on scar digestion appeared greater in CLG-4 specimen than CLG-2, while the damage to surviving myocardium was stronger in specimen of CLG-2. The maximum depth of digestion of surviving myocardium was 665.9±58.3 μm for CLG-4 while that for CLG-2 was 951±39.6 μm (p=0.004) (FIG. 1C and FIG. 1D; scale markers).

### Impact of Collagenase on Scar and Border Zone Surface Area

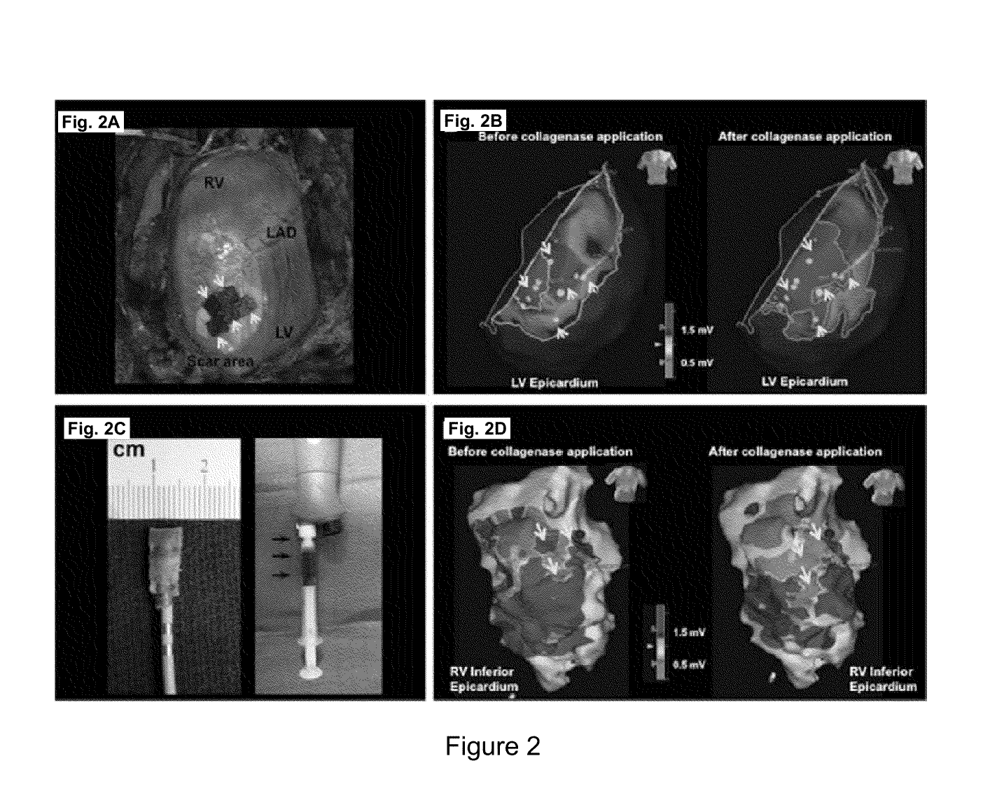

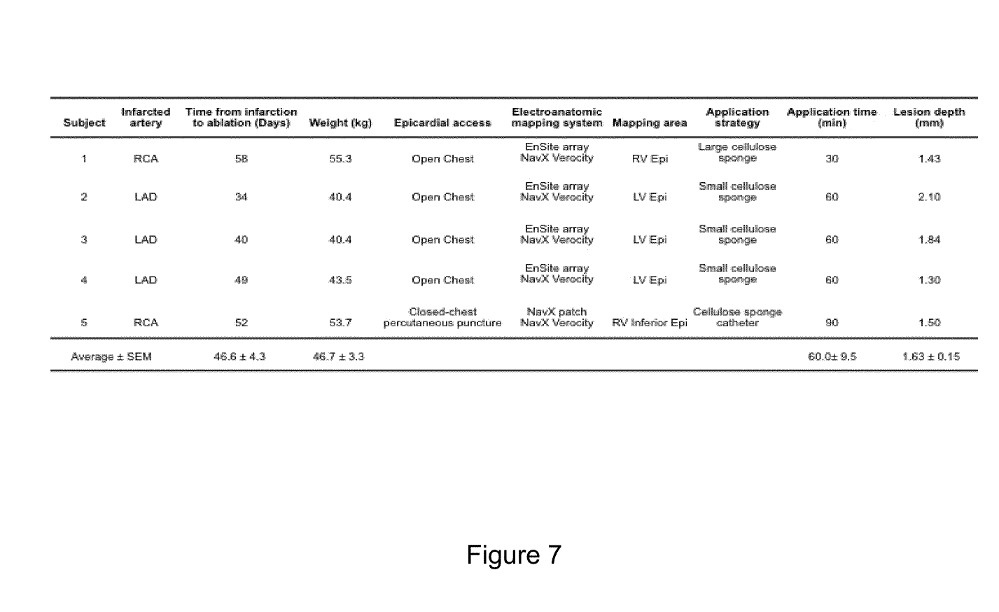

In total, four animals underwent open-chest surgical approach (FIG. 2A and FIG. 2B), while one animal underwent a closed chest surgical approach using an ablation catheter with a piece of cellulose sponge fastened onto it (0.5 cm by 1.0 cm, FIG. 2C). This allowed the catheter to be directly visible within the EAM system and selectively placed over the BZ region (FIG. 2D). CLG-4 was applied on the RV epicardium for 30 min and LV epicardium for 60 min. In subject 5, CLG-4 was applied to three different sites for 30 min each (total 90 min) because of smaller sponge size. Mean CLG-4 application time was 60.0±9.5 min across all animals, after which voltage mapping of the region was immediately repeated. The procedural details are shown in FIG. 7.

Hemodynamics were recorded continuously throughout the experiment. Systolic blood pressure and heart rate were not significantly changed before and after CLG application (68.0±7.7 versus 61.8±5.3 mmHg, P=0.08; 77.4±7.3 versus 78.8±6.0 beats/min, P=0.50, respectively).

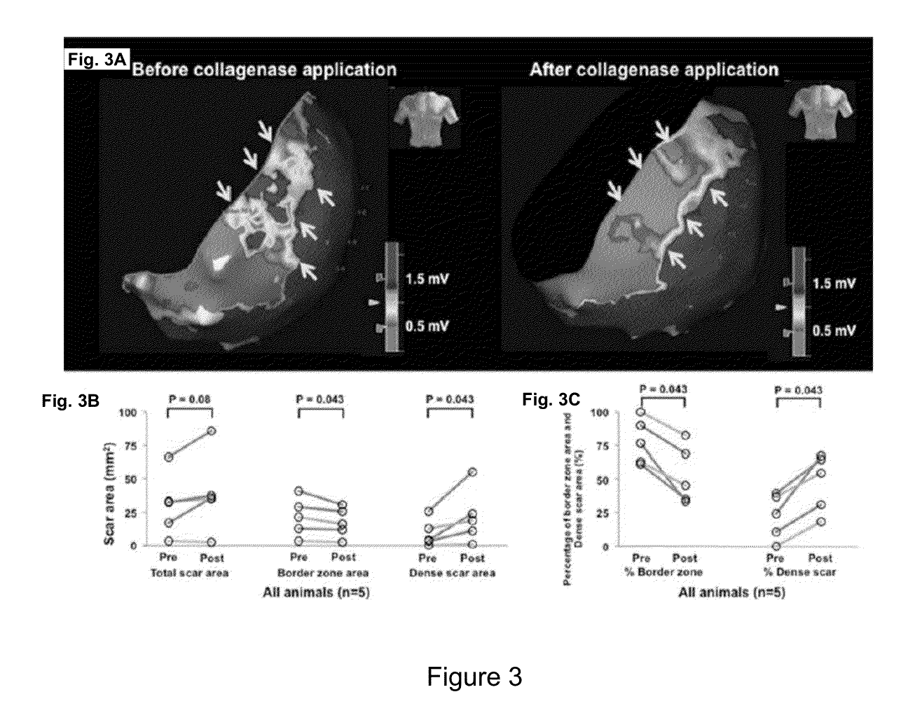

Total low voltage and DS surface areas were measured in the NavX velocity system. Regions identified as border zones were targeted for digestion with CLG-4. The total scar area before and after application of CLG-4 was 30.4±23.4 mm² and 39.2±29.5 mm² respectively, (P=0.08, n=5). Topical application of CLG-4 significantly reduced BZ surface area (21.3±14.3 mm² to 17.1±11.1 mm², P=0.043, n=5) (FIG. 3A). The reduction in BZ surface area was associated with an increase in DS surface area (9.1±10.3 mm² to 22.0±20.6 mm², P=0.043, n=5), indicating that CLG-4 application converted BZ areas to DS areas (FIG. 3B). After CLG-4 delivery, the percentage of BZ was significantly reduced, while percent DS was significantly increased (78.0±7.5% to 53.0±9.7%, and 22.0±7.5% to 47.0±9.7%, P=0.043, n=5; FIG. 3C).

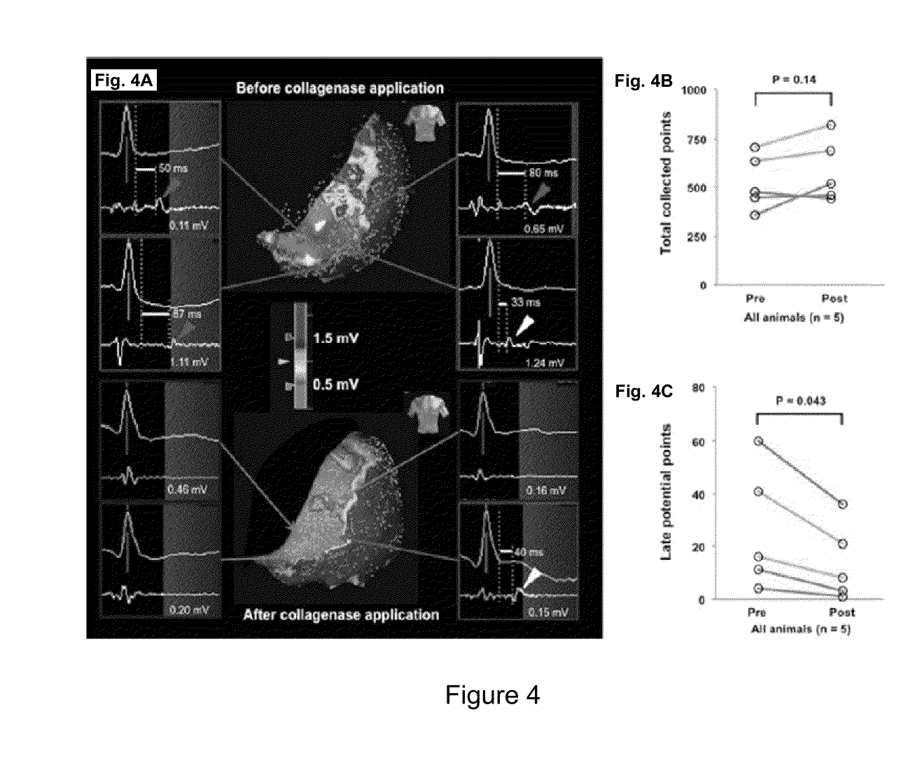

High-density mapping was performed in all animals, and LPs were quantified from all points collected. Consistent with previous studies (Nakahara et al., 2010, J Am Coll Cardiol, 55: 2355-2365) the majority of LPs were distributed in the border zone. CLG-4 application significantly reduced the number of late potentials (28.8±21.8 to 13.8±13.1, P=0.043, n=5) (FIG. 4). There was no significant difference in the total collected points pre- and post-CLG-4 application (704.5±463.0 versus 753.7±438.0, P=0.14).

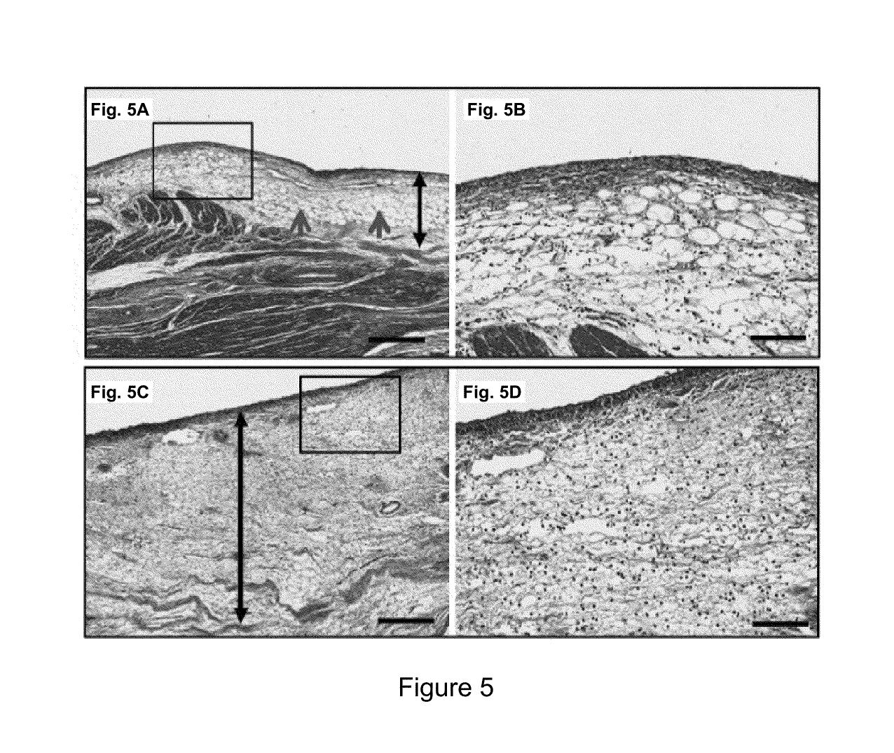

Histopathological analyses were performed after CLG-4 application in all animals. FIG. 5A and FIG. 5C show representative images of scar tissue after CLG-4 application in subjects 1 and 5, respectively. Focal debris and inflammatory changes were observed at CLG-4 application sites, while surviving myocardium distant from CLG-4 application sites remained intact. Extracellular matrix at CLG-4 application sites was degraded and appeared loosened (FIG. 5B and FIG. 5D).

### The Feasibility of Bioenzymatic Ablation of Ventricular Tissue

The major findings presented herein are (i) collagenase application to the border zones results in chemical homogenization of myocardial scars and (ii) late potentials are eradicated after the collagenase application. The experiments described herein represent the first evaluation of bioenzymatic electrical scar homogenization.

The cardiac extracellular matrix (ECM) is surrounded by myocardium, and comprises several subtypes of collagens in normal hearts (Bishop et al., 1995; Eur Heart J, 16 Suppl C: 38-44). Production of collagens induced by myofibroblasts after myocardial infarction has been described in animal models and in humans. The BZ of scars, regions where myocardial scars are adjacent to surviving myocardium represent regions of slow conduction, and sources of VT. As described in the studies presented herein, these regions were targeted for chemical ablation by CLG. The mechanism of lesion formation by CLG is the chemical interruption of tissue architecture given its inherent activity not only on collagens, but also its actions as a protease, an aminopeptidase, and a tryptase). This disruption involves both the normal and fibrotic components of the eletrophysiologically defined BZ, rendering the region electrically silent. This causes not only mechanical but also electrical disconnection of surviving myocardium, and renders the BZ region electrical silent. These plural actions result in focal ablation of the cellular and ECM components of the targeted regions.

Ablation lesions created by RF energy delivery result in tissue coagulation necrosis, infiltration of inflammatory cells, and hemorrhage in central region, which ultimately form well-demarcated lesions. These lesions also results in obliteration of cellular and ECM components in normal myocardium and scar/BZ regions. Similarly, features of these ablation lesions are also observed in lesions created with HIFU, microwave and laser technology (Haines et al., 1994, J Cardiovasc Electrophysiol, 5: 41-49). Biologic interruption of electrical conduction was previously reported, where lesions were formed by fibroblast injection to modify atrioventricular node function (Bunch et al., 2006, Circulation, 113: 2485-2494). That lesion was characterized by scar formation with collagen fibers. Others groups have performed delivery of biochemical solutions to myocardial scars by coronary artery injection or direct intramural injection (Brugada et al., 1989; Circulation, 79: 475-482; Bunch et al., 2006, Circulation, 113: 2485-2494). The study presented herein shows a novel method of epicardial scar digestion in the beating heart, involving catheters and devices already widely used clinically. Bioenzymatic ablation by CLG represents an alternate mechanism of lesion formation, which results in focal tissue destruction in regions exposed to the agent, similar to the aforementioned mechanical strategies. Topical application by open chest approach yielded focal delivery of collagenase and targeted digestion of the BZ regions only. Further, the feasibility of epicardial catheter-based delivery of CLG with focused effects on the BZ and on LPs is demonstrated herein.

The present study demonstrates electrical homogenization of myocardial scars, eliminating heterogeneous low voltage regions, and converting them into electrically silent DS. In addition, LPs, which predominantly exist in regions denoted as BZs, were significantly reduced after CLG application. Further, CLG application had no effects on LPs outside of the site of application. This is important as it indicates that the effects of CLG do not diffuse away and affect unintended sites. These findings demonstrate the focal effects at regions exposed to CLG. Although arrhythmia inducibility was not tested, significant reduction in LPs suggests reduced tissue arrhythmogenicity. Clinical studies have shown that targeting and eradicating LPs is an effective strategy for catheter-based treatment of VT (Nakahara et al., 2010, J Am Coll Cardiol, 55: 2355-2365; Di Biase et al., 2012, J Am Coll Cardiol, 60: 132-141). In the present study, CLG application was performed for 50±15.5 min at a concentration of 0.8% for CLG-4. This application time can be dramatically shortened by increasing the concentration of CLG, making it more compatible for clinical use. A higher concentration of CLG can be applied at sites of interest, and the effects terminated by α2-macroglobulin or other safe antagonists after a short period of time.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of modulating a region of cardiac tissue, comprising contacting at least a portion of the region of cardiac tissue with an effective amount of at least one therapeutic agent selected from the group consisting of a protease, a collagenase, a lipase, and a detergent.

2. The method of claim 1, wherein the method reduces the size of border zones of scar tissue, thereby at least partially homogenizing the scar tissue.

3. The method of claim 1, wherein the therapeutic agent comprises a collagenase solution.

4. The method of claim 1, wherein the collagenase acts upon type IV collagen.

5. The method of claim 1, wherein the collagenase is purified collagenase.

6. The method of claim 1, wherein the at least one therapeutic agent removes fat from the region.

7. The method of claim 1, wherein the region includes surviving myocytes.

8. The method of claim 1, wherein the region is a border zone region.

9. The method of claim 8, wherein the region is an area of low-amplitude, delayed multicomponent electric activity.

10. The method of claim 3, wherein the concentration of collagenase in the collagenase solution is between 0.001-1%.

11. The method of claim 3, wherein the amount of collagenase in the collagenase solution is between 1-1000 U/mL.

12. The method of claim 1, wherein the at least one therapeutic agent is delivered via a catheter.

13. The method of claim 12, wherein the catheter is an ablation catheter.

14. The method of claim 1, comprising using electroanatomical mapping to define the region prior to contacting at least a portion of the region with the therapeutic agent.

15. The method of claim 1, wherein the method treats arrhythmia.

16. A system for modulating a region of cardiac tissue, comprising at least one therapeutic agent selected from the group consisting of a protease, a collagenase, a lipase, and a detergent, and a device for delivering the at least one therapeutic agent to a target site of the cardiac tissue.

17. The system of claim 16, wherein the system ablates at least a portion of the region of cardiac tissue.

18. The system of claim 16, wherein the system treats arrhythmia.

19. The system of claim 16, wherein the at least one therapeutic agent comprises a collagenase solution.

20. The system of claim 19, wherein the device comprises one or more catheters and wherein the collagenase solution is delivered through a lumen of the one or more catheters.

21. The system of claim 19, wherein the collagenase solution is contained in one or more reservoirs positioned at the distal end of the device.

22. The system of claim 21, wherein the one or more reservoirs comprise an absorbent material at least partially saturated with the collagen solution.

23. The system of claim 22, wherein the absorbent material comprises a sponge or sponge like material.

24. The system of claim 21, wherein the device comprises one or more catheters connected to the one or more reservoirs for delivery of the therapeutic agent to the one or more reservoirs.

25. The system of claim 16, wherein the at least one therapeutic agent removes fat from the region.

26. An ablation kit, comprising at least one therapeutic agent selected from the group consisting of a protease, a collagenase, a lipase, and a detergent, and a device for delivering the at least one therapeutic agent to a target site of cardiac tissue.

27. The ablation kit of claim 26, wherein the at least one therapeutic agent comprises a collagenase solution.
